(19) United States
(12) Patent Application Publication
(10) Pub. No.: US 2009/0257910 A1
(43) Pub. Date: Oct. 15, 2009

(54) INTRAVENOUS CATHETER CONNECTION POINT DISINFECTION

(76) Inventor: Jeremy P. Segal, New York, NY (US)

Correspondence Address: Jeremy P. Segal, Apt. 10K, 435 East 70th St., New York, NY, 10021, US

(21) Appl. No.: 12/384,142

(22) Filed: Apr. 1, 2009

Related U.S. Application Data
(60) Provisional application No. 61/123,613, filed on Apr. 10, 2008.

(51) Int. Cl. A61L 2/08; H01J 37/20

(52) U.S. Cl. 422/22; 250/455.11

(57) ABSTRACT

A system for disinfecting an intravenous catheter and/or other fluidic tubing includes attaching a device around the catheter and delivering a high dose of ultraviolet or other type of radiation to the catheter connection point and adjacent tubing. The device may include a UV radiation source connected to a catheter attachment assembly via fiber optic cable, allowing transmission of UV radiation from the source to an internal reflector unit. The UV source may be built into the catheter connection assembly. The device may be a handheld device that fits over a catheter connection point, with single or multiple integrated UV sources to deliver 360 degree radiation around the catheter, with mirrors to increase light delivery. The method involves controlled exterior irradiation of a catheter connection point to disinfect the lumen via transmitted light through the catheter wall. Commercially available catheters or those specially designed for passage of radiation may be used.

### RELATED APPLICATIONS

This application claims priority to U.S. Provisional App. No. 61/123,613 filed Apr. 10, 2008, entitled “Intravenous Catheter Connection Point Disinfection,” which is incorporated herein by reference.

### TECHNICAL FIELD

This application is related to the field of medicine, and more particularly, to a device and method for preventing infectious complications of intravenous catheters. The device and method also has generally applicability to the sterilization of fluidics systems.

### BACKGROUND OF THE INVENTION

Infections stemming from the use of intravenous catheters are a serious problem in today's hospitals. In the United States, roughly 200,000 patients every year suffer from nosocomial infections of central venous catheters, with significant resulting morbidity and mortality. The costs are large as well, with each incidence of central venous catheter infection estimated to cost roughly $20,000.

Devices are known that address issues of disinfection/sterilization using catheters and including the use of ultraviolet (UV) or infrared radiation. For example, U.S. Pat. No. 6,461,569 to Boudreaux, entitled “Method and apparatus for ultraviolet radiation catheter sterilization system,” which is incorporated herein by reference, describes a device that utilizes a probe to be passed down the lumen of a catheter to deliver ultraviolet radiation. While this device would be able to reach colonies of bacteria resting on the tip of the catheter inside the patient, by its invasive nature it brings a risk of contamination. Additionally, such a device is presumptively designed for use following a positive diagnosis of catheter infection. Another device described in U.S. Pat. No. 5,260,020 to Wilk, et al., entitled “Method and apparatus for catheter sterilization,” which is incorporated herein by reference, describes a system for sterilization that involves a redesigned catheter designed to allow light to travel down the catheter towards the tip. This device would again be more suitably used following a positive diagnosis.

Current efforts to combat line infections center mostly on the use of universal precautions and local antisepsis during the placement of the catheter. Less emphasis is placed on ensuring antisepsis during the many times these catheters are disconnected and reconnected to switch fluids and medicines. When a patient's catheter is disconnected, the individual ends (often Luer-lock or other types of catheter connectors) are exposed to the environment, with no apparatus or method in place to ensure antisepsis. Capping with plastic caps or reattachment to a fluid source may be significant causes of contamination.

According it would be desirable to provide a system that efficiently and effectively facilitates catheter disinfection during connection and disconnection of the catheter and that, for example, prophylactically addresses the issue of exposure to the ends of the catheter during such times, as the fluid in the catheter at the connection point is confluent with the patient's bloodstream.

Maintenance of sterility in generic fluidics systems following detachment and reattachment poses similar problems. The device and method described herein, with slight modifications and alterations of scale, could be applied to a number of different fluidics applications, particularly in biotechnology.

### SUMMARY OF THE INVENTION

According to the system described herein, a device for disinfecting fluidic tubing includes a fluidic tubing attachment assembly having an internal chamber, wherein the fluidic tubing attachment assembly is securable around fluidic tubing at the point of connection. A radiation delivery assembly is coupled to the fluidic tubing attachment assembly, wherein the radiation transmission assembly transmits a bactericidal dose of ultraviolet radiation (or other type of radiation) to the internal chamber of the tubing assembly. The fluidic tubing attachment assembly may be a catheter attachment assembly securable around a catheter tubing with a central connection point. The radiation delivery assembly may include at least one fiber optic cable and may include a base radiation source coupled to the at least one fiber optic cable. The radiation may include UV radiation. The radiation delivery assembly may include a radiation source integrated with the fluidic tubing attachment assembly. The device may be a hand-held unit. The fluidic tubing attachment assembly may transmit the radiation towards the fluidic tubing. The fluidic tubing attachment assembly may further include a radiation diffuser coupled to the radiation delivery assembly that diffuses the radiation and may include an internal reflector that amplifies the radiation. The device may be sealed to prevent leakage of radiation. The radiation delivery assembly may be coupled to at least one of: a battery and a plug as a power source. A companion base station may be included that recharges the battery. The fluidic tubing may include connection point materials optimized for permeability to the radiation used by the device. A controller may control intensity of the radiation.

According further to the system described herein, a method for disinfecting fluidic tubing includes attaching a portable disinfection device around a tubing connection point. The tubing connection point is irradiated with exterior surface radiation, wherein the exterior radiation passes through the tubing connection point and directly damages microorganisms adherent to the inner surface of the catheter and microorganisms floating in lumen within the catheter. The fluidic tubing may comprise a closed catheter system or may comprise a stoppered catheter. The portable disinfection device may include a tubing attachment assembly and a radiation delivery assembly. The radiation delivery assembly may include a radiation source.

### BRIEF DESCRIPTION OF THE DRAWINGS

Embodiments of the system are described with reference to the several figures of the drawings, briefly described as follows.

FIG. 1 is a schematic illustration of a catheter sterilization device according to an embodiment of the system described herein.

FIG. 2 is a schematic illustration of a catheter sterilization device according to another embodiment of the system described herein.

### DETAILED DESCRIPTION OF VARIOUS EMBODIMENTS

Underlying the utility of the system described herein is a recognition that the act of disconnecting a catheter and reconnecting it to new bags of fluids or medications represents a source of bacterial contamination leading to nosocomial intravenous catheter infections. As further discussed elsewhere herein, the system may include a device that is designed to attach snugly around a newly reconnected catheter (or stoppered blind-ended catheter) and deliver a high dose of ultraviolet radiation (or other type of radiation) to the catheter connection point and adjacent tubing, effectively sterilizing and/or disinfecting the tubing and contained fluid at and around the catheter connection point site prior to resumption of fluid flow into the patient. The method and device of the system described herein may be used to sterilize a new connection point between fluid and catheter prior to resumption of fluid flow through the connection point. The system may also be used when a line is temporarily stoppered. Whenever a new connection is made, the device may be affixed to the outside of the catheter line at the connection point, where the device deliver a short period of high-intensity ultraviolet radiation (or other type of radiation) to the connection point, eliminating the majority of micro-organisms in and around that point. Subsequently, the flow of fluid can be restarted.

A device according to the system described herein may be attached to the outside of a sealed catheter thereby providing a significantly reduced risk for increased infections from the use of this device, as further discussed elsewhere herein. The device may be a single handheld device designed to fit over a catheter connection point with single or multiple integrated UV (or other radiation) sources to deliver 360 degree irradiation around the catheter and may include built-in mirrors to increase light delivery. The device may be designed to fit snugly around a Luer lock or similar type of catheter connection point with adjacent plastic tubing. The device holds the catheter in place within a central open chamber, positioned to allow for maximal light delivery. The device may incorporate internal mirrors to increase the delivery of ultraviolet light from all radial directions towards a catheter passing through the center of the device. A foam rubber seal may prevent escape of UV radiation.

Referring now to the figures of the drawing, the figures comprise a part of this specification and illustrate exemplary embodiments of the described system. It is to be understood that in some instances various aspects of the system may be shown schematically or may be exaggerated or altered to facilitate an understanding of the system.

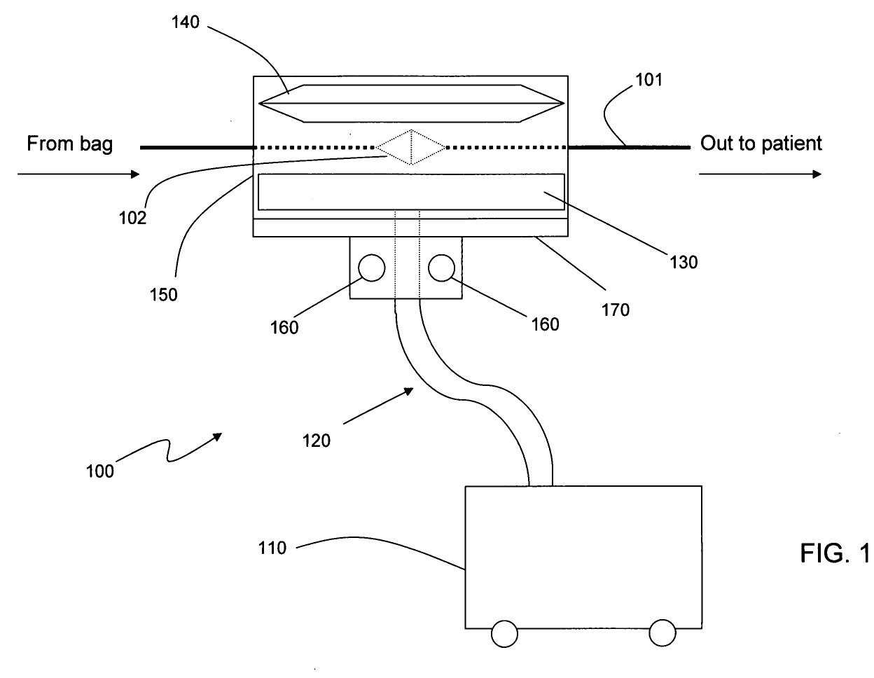

FIG. 1 is a schematic illustration of catheter sterilization device 100 according to an embodiment of the system described herein. The device 100 may include a UV radiation source 110 which may be connected to a catheter attachment assembly 150 via fiber optic transmission cable 120, allowing transmission of UV radiation from the source 110 to the catheter attachment assembly. The catheter attachment assembly 150 may be secured around a central connection point 102 of a catheter 101 between a bag and a patient. The catheter attachment assembly 150 may include a UV radiation diffuser/transmitter 130 for diffusing and/or transmitting the radiation to an internal chamber of the catheter attachment assembly 150 secured around the catheter connection point 101 and surrounding tubing. The catheter attachment assembly 150 may also include an internal reflector unit 140 for reflecting the radiation to provide amplification and further diffusion thereof. Other components 170, such as a spring hinge, may be included in the catheter attachment assembly 150 to facilitate securing of the device around the catheter. One or more controllers 160 with dials may be provided to allow user control of duration and/or intensity of the UV radiation. The one or more controllers 160 may be part of a handle for the catheter attachment assembly 150 which may be a hand-held unit. The UV radiation source 110 may be a base station that plugs directly into a wall socket. Alternatively, the radiation source 110 may include a battery unit to allow for easy portability.

As shown in FIG. 1, the segment of intravenous catheter 102 is shown with the central connection point 101 that may be, for example, of a Luer-lock or other type. Surrounding the connection point and adjacent tubing is the catheter attachment assembly that delivers the UV radiation (or other radiation type). The device holds the catheter in place within a central open chamber, positioned to allow for maximal light delivery. The catheter attachment assembly 150 may include an enclosing plastic clamp designed to fit around the tubing and allow for the internal reflectors 240 to assist with distribution of the UV radiation. The UV radiation may be generated in the attached base station 110 and transmitted to the hand-held catheter attachment assembly 150 via the fiber optic cable 120. Within the catheter attachment assembly 150, the diffuser/transmitter 130 spreads the light out to cover the entire outer surface of the catheter 102, with the assistance of the reflectors 140. The catheter attachment assembly 150 may be enclosed to prevent escape of the UV radiation. The intensity and duration of light may be adjusted by the one or more controllers 160 which may be electronic or manual dials on the device. The device 100 may be turned on by a button on the hand-held catheter attachment assembly 150, and the clamp may be maintained in place around the catheter until the entire UV dose delivery is complete. Subsequently, the device 100 may be removed and fluid flow through the catheter may be initiated.

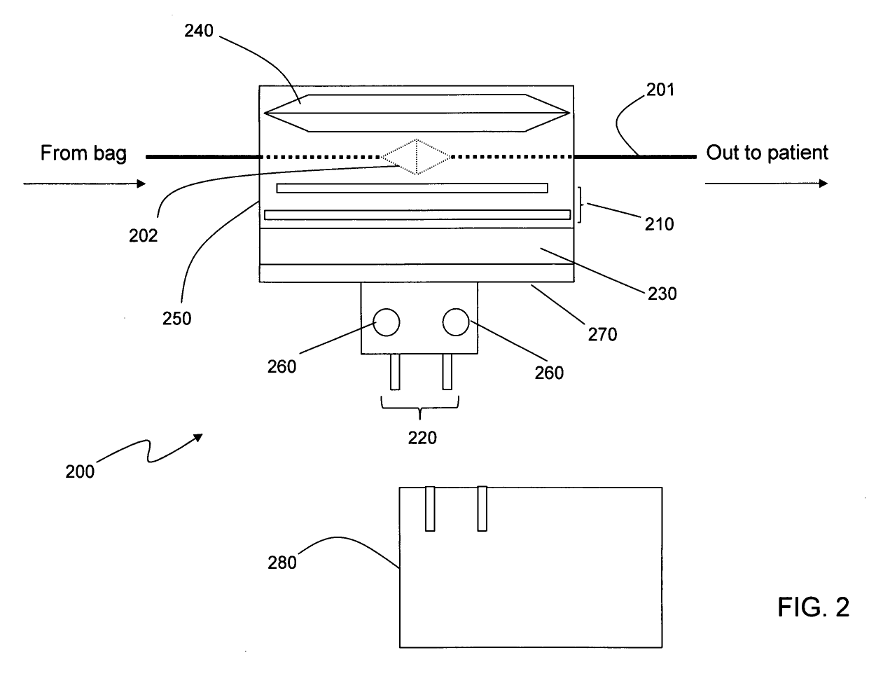

FIG. 2 is a schematic illustration of catheter sterilization device 200 according to another embodiment of the system described herein. The device 200 may include a UV radiation generator 210 that generates UV radiation and is directly coupled to and/or incorporated into a catheter attachment assembly 250 that is secured around a central connection point 202 of a catheter 201 between a bag and a patient. The UV radiation from the UV radiation source 210 transmits the radiation to internal chamber of the catheter attachment assembly secured around the catheter connection point 201 and surrounding tubing. The catheter attachment assembly 250 may also include an internal reflector unit 240 for reflecting the radiation to provide amplification and further diffusion thereof. A battery 230 may be included in the catheter attachment assembly 250 that is coupled to the UV radiation generator 210. The device 200 may include a plug 220 that allows for recharging the battery 230 when the device 200 is plugged into an external recharging base station 280. Further components 270, such as a spring hinge, may be included in the catheter attachment assembly 250 to facilitate securing of the device 200 around the catheter. One or more controllers 260 with dials may be provided to allow user control of duration and/or intensity of the UV radiation. The one or more controllers 260 may be part of a handle for the catheter attachment assembly 250 which may be a hand-held unit.

As shown in FIG. 2, instead of the use of a UV diffuser/transmitter like that of FIG. 1, a UV generator 210 may be built into the catheter attachment assembly 250 providing for a easily portable, self-contained hand-held device. The catheter attachment assembly 250 holds the catheter in place within a central open chamber, positioned to allow for maximal light delivery. The built-in UV source 210 may be powered by the self-contained battery 230. The intensity and duration of light may be selected by the one or more controllers 260 that may include electronic or manual dials on the unit, and the delivery may be initiated by a button on the handheld device. Light delivery may be augmented by the enclosed reflectors 240. The catheter connection point 201 and surrounding tubing may be bathed with high dose exterior ultraviolet light, which passes through the plastic tubing and disinfects the lumen of the catheter by a well-documented direct cytopathic effect on microorganisms. After removal of the unit and initiation of fluid flow, the hand-held device 200 may be recharged at the companion base station 280 and/or other suitable recharging mechanisms.

The system described herein may operate with a commercially-available catheter having a connection point permeable to the particular type of radiation used. Alternatively, in another embodiment, catheters specifically designed to be optimally permeable to the source radiation may be used. Accordingly, a device according to the system described herein may also incorporate a catheter and tubing specifically designed with, for example, UV permeable plastic at points of connection, with favorable geometry for optimized radiation passage.

Various embodiments discussed herein may be combined with one another in appropriate combinations. Although the system described herein is discussed principally in connection with the use of UV radiation, the system may use other types of radiation to perform sterilization. For example, the system may utilize x-rays, microwaves, ultrasound, heat and/or infra-red radiation, among others, for providing disinfection, sterilization and/or other medically-beneficial procedures in accordance with the system described herein. The system may also be used in connection with other medical devices in accordance with the devices and techniques discussed herein.

Further, the system described herein may be used in connection with fluidic tubing applications and systems other than intravenous catheters for which it may be desirable to maintain fluidics sterility at detachment and reattachment at tubing connection points. Accordingly, the descriptions and illustrations discussed herein for a catheter tubing system may be applied to other embodiments in connection with other fluidic tubing systems. Other industries for which the system described herein may be applicable include private, commercial and/or industrial systems in applications that utilize fluidic tubing, including, for example, biotech applications.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A device for disinfecting fluidic tubing, comprising:
a fluidic tubing attachment assembly having an internal chamber, wherein the fluidic tubing attachment assembly is securable around the fluidic tubing;
a radiation delivery assembly coupled to the fluidic tubing attachment assembly, wherein the radiation transmission assembly transmits radiation to the internal chamber of the fluidic tubing attachment assembly.

2. The device according to claim 1, wherein the fluidic tubing attachment assembly is a catheter attachment assembly that is securable around a catheter having a central connection point.

3. The device according to claim 1, wherein the radiation delivery assembly includes at least one fiber optic cable.

4. The device according to claim 3, wherein the radiation delivery assembly includes a base radiation source coupled to the at least one fiber optic cable.

5. The device according to claim 1, wherein the radiation is UV radiation.

6. The device according to claim 1, wherein the radiation delivery assembly includes a radiation source integrated with the fluidic tubing attachment assembly.

7. The device according to claim 6, wherein the device is a hand-held unit.

8. The device according to claim 1, wherein the fluidic tubing attachment assembly transmits the radiation towards the fluidic tubing.

9. The device according to claim 1, wherein the fluidic attachment assembly further includes a radiation diffuser coupled to the radiation delivery assembly that diffuses the radiation.

10. The device according to claim 1, wherein the fluidic attachment assembly further includes an internal reflector that amplifies the radiation

11. The device according to claim 1, wherein the device is sealed to prevent leakage of UV radiation.

12. The device according to claim 1, wherein the radiation delivery assembly is coupled to at least one of: a battery and a plug as a power source.

13. The device according to claim 10, further comprising:
a companion base station that recharges the battery.

14. The device according to claim 1, wherein the fluidic tubing includes connection point materials optimized for permeability to the radiation used by the device.

15. The device according to claim 1, further comprising:
a controller that controls intensity of the radiation.

16. A method for disinfecting fluidic tubing, comprising:
attaching a portable disinfection device around a tubing connection point;
irradiating the tubing connection point with exterior surface radiation, wherein the exterior radiation passes through the tubing connection point and directly damages microorganisms adherent to the inner surface of the fluidic tubing and microorganisms floating in lumen within the fluidic tubing.

17. The method according to claim 16, wherein the fluidic tubing comprises a closed catheter system.

18. The method according to claim 16, wherein the fluidic tubing comprises a stoppered catheter.

19. The method according to claim 16, wherein the portable disinfection device includes a fluidic tubing attachment assembly and a radiation delivery assembly.

20. The method according to claim 19, wherein the radiation delivery assembly includes a radiation source.
